(19) United States
(12) Patent Application Publication
(10) Pub. No.: US 2024/0382746 A1
(43) Pub. Date: Nov. 21, 2024

(54) SYSTEM AND METHOD FOR IMPLANTING LONGITUDINAL INTRAFASCICULAR ELECTRODES FOR HUMAN USE

(71) Applicant: BOARD OF TRUSTEES OF THE UNIVERSITY OF ARKANSAS, Little Rock, AR (US)

(72) Inventors: Anil K. Thota, Bentonville, AR (US); Ranu Jung, Fayetteville, AR (US)

(73) Assignee: BOARD OF TRUSTEES OF THE UNIVERSITY OF ARKANSAS, Little Rock, AR (US)

(21) Appl. No.: 18/668,976

(22) Filed: May 20, 2024

Related U.S. Application Data
(60) Provisional application No. 63/467,517, filed on May 18, 2023.

(51) Int. Cl. A61N 1/05

(52) U.S. Cl. CPC A61N 1/0558

(57) ABSTRACT

Embodiments of the present disclosure pertain to a system for holding at least one peripheral nerve. The system generally includes: a support surface operational for holding the peripheral nerve; a first fastener receptacle positioned on the proximal end of the support surface; and a second fastener receptacle positioned on the distal end of the support surface. Further embodiments of the present disclosure pertain to methods of holding at least one peripheral nerve by utilizing the systems of the present disclosure. Such methods generally include: placing a system of the present disclosure at or near a peripheral nerve; positioning the peripheral nerve on the support surface of the system; and placing at least one fastener on the first fastener receptacle and the second fastener receptacle of the system. The methods of the present disclosure may also include a step of implanting electrodes into or in between fascicles of the peripheral nerve.

### CROSS-REFERENCE TO RELATED APPLICATIONS

This application claims priority to U.S. Provisional Patent Application No. 63/467,517, filed on May 18, 2023. The entirety of the aforementioned application is incorporated herein by reference.

### STATEMENT REGARDING FEDERALLY SPONSORED RESEARCH

This invention was made with government support under R01-EB027584 and R01-EB023261, awarded by the National Institutes of Health. The government has certain rights in the invention.

### BACKGROUND

A need exists for more effective methods and systems for accessing fascicles of a peripheral nerve. Numerous embodiments of the present disclosure aim to address the aforementioned need.

### SUMMARY

In some embodiments, the present disclosure pertains to a system for holding at least one peripheral nerve. In some embodiments, the system includes: a support surface that is operational for holding the peripheral nerve; a first fastener receptacle positioned on the proximal end of the support surface; and a second fastener receptacle positioned on the distal end of the support surface, where each fastener receptacle is operational to receive at least one fastener.

In some embodiments, the systems of the present disclosure also include: a first angled surface positioned on the proximal end of the support surface and behind the first fastener receptacle; and a second angled surface positioned on the distal end of the support surface and behind the second fastener receptacle. In some embodiments, the systems of the present disclosure also include a third fastener receptacle positioned on a first side of the support surface, and a fourth fastener receptacle positioned on a second side of the support surface, where each fastener receptable is operational to receive at least one fastener. In some embodiments, the first and the second side are on opposite sides of one another.

The systems of the present disclosure may also include an aperture positioned below the support surface and spanning the width of the support surface. In some embodiments, the aperture is operational to receive a cord for lifting the system. In some embodiments, the systems of the present disclosure also include a base area positioned below the support surface for stabilizing the system on a surface.

Additional embodiments of the present disclosure pertain to methods of holding at least one peripheral nerve by utilizing the systems of the present disclosure. Such methods generally include: placing a system of the present disclosure at or near a peripheral nerve; positioning the peripheral nerve on the support surface of the system; and placing at least one fastener on the first fastener receptacle and the second fastener receptacle of the system.

In some embodiments, the methods of the present disclosure may be utilized to facilitate access to one or more fascicles of a peripheral nerve. In some embodiments, the methods of the present disclosure also include a step of implanting one or more electrodes into or in between one or more fascicles of the peripheral nerve after positioning the peripheral nerve on the support surface.

In some embodiments, the methods of the present disclosure occur in vitro. In some embodiments, the methods of the present disclosure occur in vivo in a subject, such as a human being.

### BRIEF DESCRIPTION OF THE DRAWINGS

A better understanding of the present invention can be obtained when the following detailed description is considered in conjunction with the following drawings, in which:

FIG. 1 illustrates a system for holding peripheral nerves in accordance with various embodiments of the present disclosure. Such systems may also be referred to herein as a reinforcing longitudinal intrafascicular electrode-implantation facilitator (ReLIFT). Versions of ReLIFT are shown in FIG. 6B, FIGS. 7A-7B and FIGS. 8A-8C.

FIGS. 2A-2B illustrate the design and use of a longitudinal intrafascicular electrode-implantation facilitator (LIFT) system. FIG. 2A shows images of successfully transferred and fabricated LIFT designs for different nerve sizes with the Formlabs Form 3 three-dimensional (3D) printer using biocompatible BioMed White Resin. FIG. 2B shows the in vivo implementation of the LIFT system in a rat.

FIGS. 3A-3C show data related to the stress evaluations of LIFT systems. Shown are von Mises stress profiles when a surgeon holds the LIFT with a surgical instrument at the fastener receptacles on the ledge.

FIGS. 4A-4C show additional data related to the stress evaluations of LIFT systems. Shown are von Mises stress profiles when a surgeon holds the LIFT with a surgical instrument at one of the arms on the reshaping fastener receptacles.

FIGS. 5A-5C provide data related to an in vivo rabbit study showing the feasibility of scaling the dimensions of the LIFT and using it to implant longitudinal intrafascicular electrodes (LIFEs) in a large diameter nerve. FIG. 5A shows the implementation of LIFT in a rabbit sciatic nerve. FIG. 5B shows strength duration curves from the LIFEs in tibial and peroneal fascicles. FIG. 5C shows ankle angle data assessments. The data show that stimulating the peroneal fascicle activates the tibialis anterior (TA) muscle, eliciting ankle flexion (decreasing angle: ankle moving upwards). Stimulating tibial fascicle activates the gastrocnemius muscle (GM), eliciting ankle flexion (increasing angle: ankle moving downwards).

FIGS. 6A-6B show different LIFT designs, including the original LIFT (FIG. 6A) and a modified reinforcing LIFT (ReLIFT) (FIG. 6B), where reinforcing fastener receptacles were added to sustain the forces applied by the vessel loops to retain the reshaped form of a nerve.

FIGS. 7A-7B show data related to the clinical validation of ReLIFT in cadaver tissue. FIG. 7A shows the application of ReLIFT to large animal model (i.e., a pig). FIG. 7B shows vessel loop alternatives for the LIFT design targeting a range of nerve sizes.

FIGS. 8A-8C illustrate a simulated clinical implementation of ReLIFT. FIG. 8A shows an image of surgeons implementing ReLIFT in a human cadaver preparation. FIG. 8B shows the use of reinforcement fastener receptacles of ReLIFT to secure and reshape a nerve. Preloading the vessel loops in the reinforcement fastener receptacles improved the case of implementation. FIG. 8C shows threading of needles for implanting electrodes after setting up the ReLIFT.

### DETAILED DESCRIPTION

It is to be understood that both the foregoing general description and the following detailed description are illustrative and explanatory, and are not restrictive of the subject matter, as claimed. In this application, the use of the singular includes the plural, the word “a” or “an” means “at least one”, and the use of “or” means “and/or”, unless specifically stated otherwise. Furthermore, the use of the term “including”, as well as other forms, such as “includes” and “included”, is not limiting. Also, terms such as “element” or “component” encompass both elements or components comprising one unit and elements or components that include more than one unit unless specifically stated otherwise.

The section headings used herein are for organizational purposes and are not to be construed as limiting the subject matter described. All documents, or portions of documents, cited in this application, including, but not limited to, patents, patent applications, articles, books, and treatises, are hereby expressly incorporated herein by reference in their entirety for any purpose. In the event that one or more of the incorporated literature and similar materials defines a term in a manner that contradicts the definition of that term in this application, this application controls.

The human peripheral nervous system (PNS) facilitates communication between the organs and glands in the body and the central nervous system (CNS). Bidirectional communications include the PNS relaying motor commands from the CNS to the voluntary muscles for the movement of limbs and sending sensory information, such as touch and pressure, from the body's sensory organs to the CNS. This forms the somatic nervous system (SNS) component of the PNS.

The PNS communicates CNS signals that assert involuntary control of bodily functions, such as blood pressure, breathing, gut motility, urination, vision, and sweating by controlling several other body organs and glands, such as the heart, airways, stomach, bladder, pupils, and saliva. This forms the autonomic nervous system (ANS) component of the PNS.

Pain is also linked with the autonomic nervous system. Communication between the PNS and the CNS gets disrupted when degeneration, damage, or severing of nerve fibers and nerves occurs under certain disease or nerve trauma conditions. Disrupted communication leads to the loss of sensory, motor, autonomic, or a combination of one or more functions.

Peripheral nerve fiber activation, using techniques such as electrical stimulation, is being utilized to re-establish communication of the CNS with the PNS. Such peripheral neuromodulation has been used to restore lost sensory functions in the extremities of individuals with lower and upper limb amputations, control neuropathic pain, relieve urinary bladder incontinence, and address many other debilitating conditions.

Since the first description in the seventeenth century of the utilization of neural stimulation using peripheral neural interfaces (PNI) to elicit function, multiple researchers have used multiple types of peripheral nerve electrode interfaces to establish connectivity between the electrode and peripheral neural tissue to achieve the requisite neuromodulation, or to record the activity of nerve fibers.

Mammalian peripheral nerves consist of one or more nerve fascicles enveloped in epineurial tissue. The oligofascicular nerve consists of 2-10 fascicles and polyfascicular nerves contain more than 10 fascicles. Each fascicle includes groups of individual myelinated and/or unmyelinated nerve fibers wrapped in perineurial tissue.

The most used PNI electrodes are nerve cuff electrodes. Cuff electrodes are wrapped around the nerve and have been used for over three decades. Injectrodes, which can be placed adjacent to the nerve, are alternatives to cuff electrodes that have been recently developed. To achieve close contact with nerve fibers, however, intraneural electrodes, and in particular intrafascicular electrodes, have to be used.

The selectivity of an electrode is defined as the ability to interface with a distinct group of nerve fibers. Since the extensively used cuff electrodes are wrapped around the nerve and the injectrode is adjacent to the nerve, they are both farther from the nerve fibers inside the fascicles and hence typically have lower selectivity. Selectivity can be increased by placing the electrodes near the target nerve fibers.

Thus, an intrafascicular electrode that penetrates nerve fascicles can interface directly with the target nerve fibers and hence promises higher selectivity. The highly selective electrode can be used in neuromodulation approaches to directly influence the activity of distinct groups of nerve fibers surrounding the electrode.

Neural electrodes designed to have high selectivity and specificity, such as intrafascicular electrodes, which can be utilized to achieve optimal therapeutic outcomes, may be especially desirable for use in an emerging neuromodulation research field, bioelectronic medicine. Bioelectronic medicine promises to improve and restore health without the debilitating side effects of systemically administered drugs by modulating neural communication between the brain and the end organs affected by diseases.

Currently, PNIs, primarily cuff electrodes, are being used to stimulate nerves for treating diabetes, inflammation, rheumatoid arthritis, and many other disorders or disease conditions. For achieving ideal goals for bioelectronic medicine applications, which would benefit from reduced off-target activation of nerve fibers, inevitable with cuff and injectrode type PNIs, the following PNI characteristics may be relevant: 1) The PNI permits accessing a small group of fibers within each nerve fascicle; 2) The PNI offers reliable recording or stimulation of the fibers over the PNI's lifespan, and 3) the PNI does not harm the host nerve.

Intrafascicular electrodes offer the aforementioned characteristics. Although more invasive to the nerve than nerve cuff electrodes, intrafascicular electrodes offer more selectivity in achieving the intended nerve stimulation response and hence are ideal neural electrodes for bioelectronic medicine applications. In either completed or ongoing human research/clinical studies to interface with the somatic nervous system, three types of intrafascicular electrodes have been utilized: 1) Utah Slanted Electrode Array (USEA), 2) Transverse Intrafascicular Multichannel Electrode (TIME), and 3) Longitudinal Intrafascicular Electrodes (LIFE).

The LIFE offers high selectivity as well as good mechanical compatibility with the peripheral nerve into which it is implanted because each electrode is fabricated with a highly flexible insulated microwire. A tungsten needle (or other introducer) is attached at one end to insert the wire into the fascicle so that the active contact area for delivering electrical charge lies longitudinally within the fascicle parallel to the nerve fibers. The needle (or introducer) is discarded after the implantation. The longitudinal placement of the microwire parallel to the nerve fibers is also compatible with the inevitable stretching of peripheral nerves as the body moves.

LIFEs have been implanted in human participants in research studies. For increasing selectivity, multiple LIFEs can be implanted in one or more fascicle within a nerve. To permit management of these fine microwires, which could easily get entangled and damaged during the surgical implantation procedure, Applicant previously developed a distributed intrafascicular multi-electrode (DIME) lead consisting of multiple LIFEs packaged in a single lead, and a multi-lead multi-electrode (MLME) system to facilitate implanting multiple DIMEs in multiple nerves.

The MLME system permits implantation of multiple LIFEs to access multiple nerves and fascicles while reducing risks of fine wire breakage and entanglement. However, the surgical implantation approach for maximizing selectivity and specificity by targeting multiple fascicles offers challenges, especially to surgeons not accustomed to routine peripheral nerve surgery.

For instance, some fascicles in a polyfascicular nerve are not easily accessible for electrode implantation because the nerve is oval. Another reason why polyfascicular nerves are not easily accessible is because the nerve can readily buckle during implantation because the nerve is a compliant structure. Implantation of multiple electrodes further increases implantation complexity and time to implant.

As such, a need exists for more effective methods and systems for accessing fascicles of a peripheral nerve. Numerous embodiments of the present disclosure aim to address the aforementioned need.

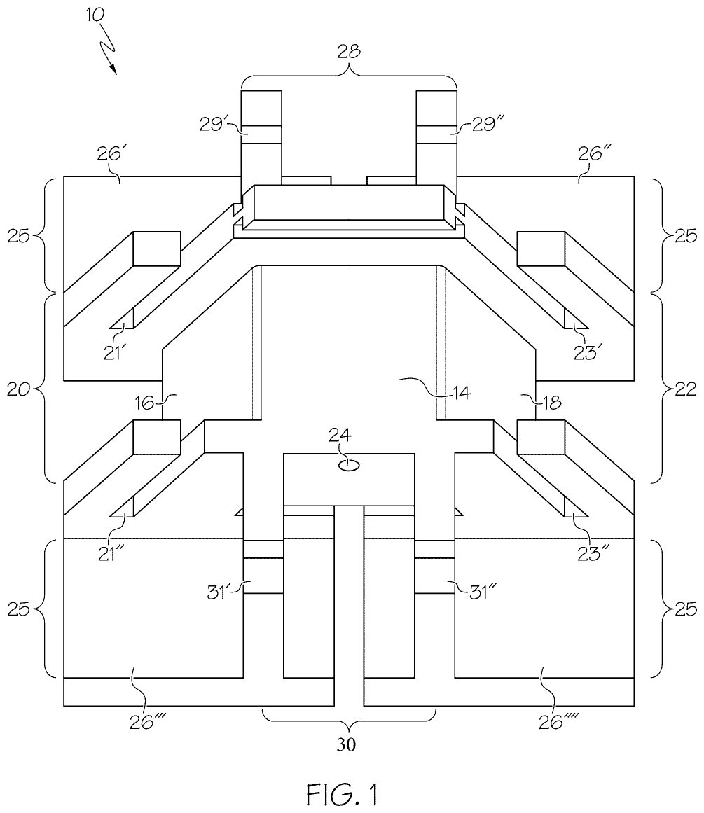

In some embodiments, the present disclosure pertains to a system for holding at least one peripheral nerve. For illustrative purposes, the systems of the present disclosure may be depicted as system 10 in FIG. 1. System 10 generally includes a support surface 14 that is operational for holding a peripheral nerve, a first fastener receptacle 20 positioned on the proximal end of support surface 14, and a second fastener receptacle 22 positioned on the distal end of support surface 14. In some embodiments, each of fastener receptacles 20 and 22 are operational to receive at least one fastener. Additionally, each of fastener receptacles 20 and 22 may include a plurality of slots for receiving at least one fastener. For instance, fastener receptacle 20 may include slots 21′ and 21″. Likewise, fastener receptacle 22 may include slots 23′ and 23″.

As further illustrated in FIG. 1, system 10 may also include a first angled surface 16 positioned on the proximal end of support surface 14 and behind the first fastener receptacle 20, and a second angled surface 18 positioned on the distal end of support surface 18 and behind the second fastener receptacle 22. In some embodiments further illustrated in FIG. 1, system 10 may also include an aperture 24 positioned below support surface 14. In some embodiments, aperture 24 spans the width of support surface 14. In some embodiments, aperture 24 is operational to receive a cord for lifting system 10.

As further illustrated in FIG. 1, system 10 may also include a base area 25 positioned below support surface 14. In some embodiments, base area 25 is operational to stabilize system 10 on a surface. In some embodiments further illustrated in FIG. 1, base area 25 may further include a plurality of base sub-components 26′, 26″, 26′″, and 26″″.

In some embodiments further illustrated in FIG. 1, system 10 also includes a third fastener receptacle 28 positioned on a first side of support surface 14, and a fourth fastener receptacle 30 positioned on a second side of support surface 14. In some embodiments, the first and the second sides are on opposite sides of one another. In some embodiments, each of fastener receptacles 28 and 30 are operational to receive at least one fastener. Additionally, each of fastener receptacles 28 and 30 may include a plurality of slots for receiving at least one fastener. For instance, fastener receptacle 28 may include slots 29′ and 29″. Likewise, fastener receptacle 30 may include slots 31′ and 31″.

Additional embodiments of the present disclosure pertain to methods of utilizing the systems of the present disclosure to hold at least one peripheral nerve. With reference again to system 10 in FIG. 1 for illustrative purposes, the methods of the present disclosure include: placing system 10 at or near a peripheral nerve; positioning the peripheral nerve on support surface 14; and placing at least one fastener on first fastener receptacle 20 and second fastener receptacle 22. In some embodiments, the same fastener may be placed on first fastener receptacle 20 and second fastener receptacle 22. In some embodiments, different fasteners may be placed on first fastener receptacle 20 and second fastener receptacle 22.

In some embodiments, the methods of the present disclosure also include placing at least one fastener on third fastener receptacle 28 and fourth fastener receptacle 30. In some embodiments, the same fastener may be placed on third fastener receptacle 28 and fourth fastener receptacle 30. In some embodiments, different fasteners may be placed on third fastener receptacle 28 and fourth fastener receptacle 30. In some embodiments, the methods of the present disclosure also include applying a cord through aperture 24 and lifting system 10 by pulling on the cord.

As set forth in more detail herein, the systems of the present disclosure may have various structures and arrangements. Additionally, the methods of the present disclosure may be utilized to hold various peripheral nerves in various arrangements and for various purposes.

### Support Surfaces

The systems of the present disclosure may include various support surfaces. For instance, in some embodiments, the support surface includes a flat surface. In some embodiments, the support surface includes a rectangular shape. In some embodiments, the support surface represents a non-angled and flat surface.

### Fastener Receptacles

The systems of the present disclosure may include various types of fastener receptacles. For instance, in some embodiments, the systems of the present disclosure include a first fastener receptacle (e.g., fastener receptable 20 illustrated in FIG. 1) positioned on the proximal end of a support surface, and a second fastener receptacle (e.g., fastener receptable 22 illustrated in FIG. 1) positioned on the distal end of a support surface. In some embodiments, the first and second fastener receptacles are each operational to sustain tension on a peripheral nerve from a fastener.

In some embodiments, the systems of the present disclosure also include a third fastener receptacle (e.g., fastener receptacle 28 illustrated in FIG. 1) positioned on a first side of a support surface, and a fourth fastener receptacle (e.g., fastener receptacle 30 illustrated in FIG. 1) positioned on a second side of a support surface. In some embodiments, the first and the second side are on opposite sides of one another. In some embodiments, the third and the fourth fasteners are operational to stabilize the systems of the present disclosure on a surface (e.g., a surface at or near a peripheral nerve).

In some embodiments, each fastener receptacle protrudes outward from a system of the present disclosure. In some embodiments, each fastener receptacle is in the form of a reinforcing anchor. In some embodiments, each fastener receptacle includes one or more slots operational to receive a fastener (e.g., slots 21, 23, 29, and 31 illustrated in FIG. 1).

The fastener receptacles of the present disclosure may be operational to receive various types of fasteners. For instance, in some embodiments, the fasteners include biocompatible fasteners. In some embodiments, the fastener is in the form of a rope. In some embodiments, the fastener is in the form of a molded silicone, a plastic flap, or a silicone tube. In some embodiments, the fastener is in the form of a vessel loop, such as a vessel loop routinely used in vascular surgeries.

Fasteners may be applied to fastener receptacles in various manners. For instance, in some embodiments, the application includes applying a force onto the fastener. In some embodiments, the applied force keeps a peripheral nerve in place and flattened out.

The positioning of fasteners on fastener receptacles may have various effects. For instance, in some embodiments, the placing of at least one fastener on the first fastener receptacle and the second fastener receptacle secures the peripheral nerve on the system. In some embodiments, placing of at least one fastener on the first fastener receptacle, the second fastener receptacle, the third fastener receptacle, and the fourth fastener receptacle secures the peripheral nerve on the system.

### Apertures

In some embodiments, the systems of the present disclosure may also include an aperture that is positioned below the support surface (e.g., aperture 24 below support surface 14, as illustrated in FIG. 1). In some embodiments, the aperture spans the width of the support surface. In some embodiments, the aperture is in the form of a circular hole. In some embodiments, the aperture is operational to receive a cord for lifting the system.

In some embodiments, the methods of the present disclosure also include a step of placing a cord through an aperture and lifting the system by pulling on the cord. In some embodiments, the lifting occurs after placing a peripheral nerve on a support surface.

### Holding of Peripheral Nerves

The methods of the present disclosure may utilize the systems of the present disclosure to hold peripheral nerves in various manners. For instance, in some embodiments, a system of the present disclosure may be placed on a tissue at or near a peripheral nerve.

In some embodiments, a peripheral nerve may be positioned on a support surface by pulling the peripheral nerve from its native environment. In some embodiments, the positioning of the peripheral nerve on a support surface includes isolating the peripheral nerve from its native environment, such as a connective tissue, muscle fascia, adipose tissue, and/or blood vessels. In some embodiments, the positioning of the peripheral on a support surface occurs prior to placing at least one fastener on the first fastener receptacle and the second fastener receptacle of a system of the present disclosure. In some embodiments, the positioning of the peripheral on a support surface occurs prior to placing at least one fastener on the first fastener receptacle, the second fastener receptacle, the third fastener receptacle, and the fourth fastener receptacle of a system of the present disclosure.

The methods of the present disclosure may be utilized to hold various types of peripheral nerves. For instance, in some embodiments, the peripheral nerve includes, without limitation, a brachial plexus, a peroneal nerve, a femoral nerve, a lateral femoral cutaneous nerve, a sciatic nerve, a spinal accessory nerve, a tibial nerve, an autonomic nerve, branches thereof, or combinations thereof. In some embodiments, the peripheral nerve includes an autonomic nerve.

### Effects on Peripheral Nerves

The methods and systems of the present disclosure may have various effects on peripheral nerves. For instance, in some embodiments, the methods and systems of the present disclosure can be used to immobilize a peripheral nerve on a support surface.

In some embodiments, the methods and systems of the present disclosure can be used to reshape a peripheral nerve. In some embodiments, the reshaping includes reshaping the peripheral nerve to a flat structure. In some embodiments, the reshaping includes aligning the fascicles of the peripheral nerve. In some embodiments, the reshaping includes reshaping the peripheral nerve to an elongated ellipse. In some embodiments, peripheral nerve fascicles become distributed across the axis of the elongated ellipse. In some embodiments, the peripheral nerve fascicles become distributed on the surface of the elongated ellipse.

In some embodiments, the methods and systems of the present disclosure can be used to facilitate access to one or more fascicles of a peripheral nerve. In some embodiments, the methods and systems of the present disclosure can be used to facilitate access to fascicles of a peripheral nerve for implantation of one or more electrodes (e.g., intrafascicular or other intraneural electrodes) into or in between the fascicles of the peripheral nerve.

### Implantation of Electrodes

In some embodiments, the methods of the present disclosure also include a step of implanting one or more electrodes into or in between one or more fascicles of a peripheral nerve. In some embodiments, the implantation occurs while the peripheral nerve is being held by a system of the present disclosure.

Electrodes may be implanted into or in between various fascicles of a peripheral nerve. For instance, in some embodiments, the fascicles include, without limitation, a sural fascicle, a peroneal fascicle, a tibial fascicle, a sensory fascicle, a motor fascicle, a mixed fascicle, a nerve branch thereof, or combinations thereof.

The methods of the present disclosure may implant various electrodes into or in between peripheral nerves. For instance, in some embodiments, the electrodes include, without limitation, neural electrodes, intraneural electrodes, intrafascicular electrodes, or combinations thereof. In some embodiments, the electrodes include intrafascicular electrodes. In some embodiments, the intrafascicular electrodes include, without limitation, a longitudinal intrafascicular electrode (LIFE), a thin film longitudinal intrafascicular electrode (tf-LIFE), a poly longitudinal intrafascicular electrode (polyLIFE), a transverse intrafascicular multichannel electrode (TIME), a Utah slant electrode array (USEA), a distributed intrafascicular multielectrode (DIME), or combinations thereof.

### Modes of Operation

The methods and systems of the present disclosure may be operated in various modes. For instance, in some embodiments, the methods and systems of the present disclosure may be utilized to hold peripheral nerves in vitro. In some embodiments, the methods and systems of the present disclosure may be utilized to hold peripheral nerves in vivo in a subject. In some embodiments, the subject is a human being. In some embodiments, the subject is a non-human mammal. In some embodiments, the non-human mammal includes, without limitation, a horse, a rabbit, a mouse, a rat, a pig, a sheep, a cow, a dog, or a cat. In some embodiments, the non-human mammal is a domestic animal, such as a dog, a cat, or a nonhuman primate.

### Applications and Advantages

The methods and systems of the present disclosure can have numerous advantages. For instance, in some embodiments, the methods and systems of the present disclosure can be used to enhance the implantation selectivity of numerous electrodes. Additionally, in some embodiments, the methods and systems of the present disclosure can help reduce the invasiveness of electrode implantation procedures by at least (1) avoiding extensive epineurial dissection to identify the fascicles; (2) reducing the complexity of an implantation process by stabilizing peripheral nerves; (3) reducing the time for implantation of multiple electrodes; and (4) reducing peripheral nerve trauma during the electrode implantation process.

In some embodiments, the methods and systems of the present disclosure can also help streamline the electrode implantation process. In some embodiments, the methods and systems of the present disclosure can help manage surgical space by isolating peripheral nerves from surroundings during electrode implantation.

In some embodiments, the methods and systems of the present disclosure reduce a need for surgical training and specialization of surgeons. As such, in some embodiments, the methods and systems of the present disclosure can make peripheral nerve implants for bioelectronic medicine more accessible.

In some embodiments, the methods and systems of the present disclosure may be utilized to repair a peripheral nerve. As such, in some embodiments, the methods of the present disclosure may also include a step of repairing a peripheral nerve. For instance, in some embodiments, the methods and systems of the present disclosure may be utilized to repair a damaged peripheral nerve. In some embodiments, the methods of the present disclosure may be used to align the fascicles in repairing completely or partially lacerated nerves. In some embodiments, a step of repairing a peripheral nerve occurs independently of, or in lieu of, a step of implanting an electrode. In some embodiments, a step of repairing a peripheral nerve occurs without an electrode implantation step.

### Additional Embodiments

Reference will now be made to more specific embodiments of the present disclosure and experimental results that provide support for such embodiments. However, Applicant notes that the disclosure below is for illustrative purposes only and is not intended to limit the scope of the claimed subject matter in any way.

### Example 1

### Design and Validation of ReLIFT

This example describes the fabrication process, choice of fabrication material, and implementation procedure for a longitudinal intrafascicular electrode-implantation facilitator (LIFT) and a reinforced version thereof (ReLIFT), systems for stabilizing and/or reshaping peripheral nerves. Applicant developed ReLIFT as an improved system for implanting electrodes in a subject's peripheral nerves.

### Example 1.1.

### LIFT Design, Fabrication, and Description

A goal of designing, developing, and testing the LIFT was to assist surgeons in the implantation process of intraneural electrodes, such as longitudinal intrafascicular electrodes (LIFEs), distributed intrafascicular multielectrodes (DIMEs), and other similar intraneural electrodes in the targeted nerve. The three-dimensional models of the LIFT were developed and fabricated using a commercial three-dimensional printer. In in-vivo rat preparations, Applicant has validated the functionality of the LIFT as well as developed the instructions for use (IFU) of LIFT. Such instructions include the following steps: (1) pass a vessel loop through a stabilization slot and place the LIFT next to the nerve; (2) slip the nerve onto the LIFT's platform and into its channels using micro forceps while aligning and straightening the nerve with the micro forceps; (3) slide commercially available vessel loops or custom silicone inserts into reshaping slots using micro forceps; (4) stabilize and manipulate the LIFT's angle by holding the ledge with micro forceps; and (5) hold a needle (LIFE introducer) with a microneedle holder without a lock, thread the needle into the fascicle parallel to the nerve fibers, and grab the needle tip pierced out of the fascicle and pull the electrode into the fascicle. Steps 4 and 5 can be used to implant as many LIFEs as needed.

### Example 1.2

### Intended use of LIFT and ReLIFT

A proposed use of LIFT and ReLIFT is a single-use, sterile kit intended to assist surgeons in the implantation process of intraneural electrodes, such as LIFE, DIME, and other similar intraneural electrodes. The intraneural electrodes can be used to stimulate (or record from) peripheral nerves in any part of the human body for therapeutic, diagnostic, and preventive applications in the field of electroceuticals™, bioelectronic medicine, and neuromodulation therapies. The sterile kit includes the appropriate sizes of LIFT or ReLIFT, vessel loops, re-usable micro needle holders, and other approved accessories.

### Example 1.3

### Design of LIFT

LIFT was designed from the following two primary design requirements to reduce electrode implantation time and complexity and increase fascicular access: 1) to support and secure the stabilization of the nerve, and 2) to reshape the nerve from an oval to an elongated ellipse.

LIFT can assist surgeons by supporting and reshaping the target nerve. An appropriately sized flat platform for placing the nerve acts as a nerve supporter. The nerve supporter feature stabilizes the nerve during the implantation of multiple LIFEs or DIMEs. The nerve shaper reshapes the nerve from an oval to an elongated ellipse. The reshaped nerve is retained by securing the nerve to the nerve supporter. The reshaped nerve increases the access to fascicles for implanting LIFEs.

### Example 1.4

Use of LIFT with Intraneural Electrodes

In addition to LIFE and DIMEs, Utah slanted electrode arrays (USEA) and transverse intrafascicular multichannel electrodes (TIME) are the other two intrafascicular electrodes that are being used in human research and clinical studies. The USEA electrode arrays are inserted into the nerve using a pneumatic electrode inserter. TIME electrode arrays are threaded into the nerve transversally with the needle attached to the electrode array with a suture. The USEA and TIME electrode arrays are single implantable units with multiple electrodes that do not need to stabilize the nerve during the implantation process. However, the DIME electrode array includes multiple individual LIFEs fabricated using a highly flexible insulated wire (˜25 μm diameter) implanted with a tungsten needle. Stabilization of the nerve is imperative to prevent the compliant nerve from buckling during the implantation of LIFEs. In the current DIME implantation, trained surgeons use their preferred methods, such as using vessel loops, fine forceps, or sterile tubes, to stabilize the nerve to implant multiple LIFEs.

The nerve supporter feature was incorporated into the LIFT design to help facilitate the LIFE implantation by stabilizing the nerve. The neural interface is effective and highly functional if all or most of the fascicles in a nerve are accessible by the interface. Accessing fascicles in the middle of a polyfascicular or oligofascicular nerve is challenging because the nerve is naturally circular where fascicles are circumferentially distributed.

In the current DIME implantation, trained surgeons use their microsurgical skills to carefully dissect the epineurium to access the fascicles. The nerve shaper feature was incorporated into the LIFT design to help increase access to the fascicles. The nerve shaper distributes the fascicles across the major axis of the elongated ellipse. The reshaped nerve brings the fascicles in the middle of the nerve to the surface of the ellipse. In addition, the nerve shaper mechanism is attached to the nerve supporter to secure the nerve in place during implantation.

### Example 1.5

### LIFT Design and Manufacturing

The LIFT prototypes for in-vivo rodent studies were fabricated using a commercial three-dimensional (3D) fused deposition modeling printer (Ender V2, Creality, China). For human clinical use, the units can be fabricated with a known biocompatible material that can be sterilized.

Applicant has identified a Form of 3D printer (Formlabs Inc., MA, USA) to fabricate scaled LIFT versions for clinical use with their BioMed White Resin. BioMed White Resin is a medical-grade biocompatible (ISO 10993-5, 10, 11, and USP<151>) resin. The deployable LIFT units can be fabricated, packaged, and sterilized at an FDA-registered contract manufacturing facility. The contract manufacturer can follow the 3D printer manufacturer-validated process to segment the 3D models, print, post-process (wash, dry, and cure), verify and sterilize. Following a manufacturer's validated process can ensure the sterilizability and biocompatibility of the printed units to be deployed for human use.

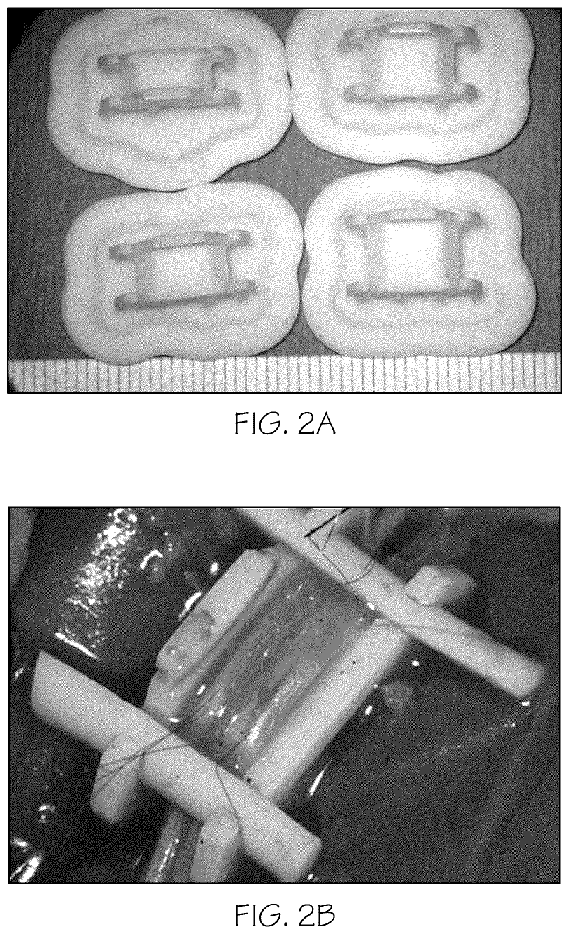

Applicant has successfully printed LIFT prototypes with a Form 3 printer using biocompatible BioMed White Resin. FIG. 2A shows 3D-printed units with a Form 3 printer. FIG. 2B shows the implementation of the unit in in-vivo rat preparation.

### Example 1.6

### LIFT Physical Performance Characteristics

The physical performance characteristics of the LIFT can provide support to safely secure the nerve to facilitate electrode implantation processes. LIFT can provide sufficient stiffness to support the nerve and have a mechanism to secure the nerve without damaging it. Additionally, LIFT can be easy to deploy in various surgical environments for implantation processes.

The LIFT units for in-vivo validation were 3D printed using polylactic acid (PLA) filament, and the units for human deployment can be fabricated using BioMed White Resin. The young's modulus, a stiffness property of BioMed White Resin, is 2020 MPa. On the other hand, the young's modulus of human nerves is approximately 20 MPa. Hence, LIFT can support the nerve during an implantation process.

The 3D printed units with PLA were successfully used in the in-vivo rodent validation studies, proving that the tool facilitated the implantation of the electrodes. The results show that the LIFT was easily deployed in the surgical environment and reduced the time to implant electrodes.

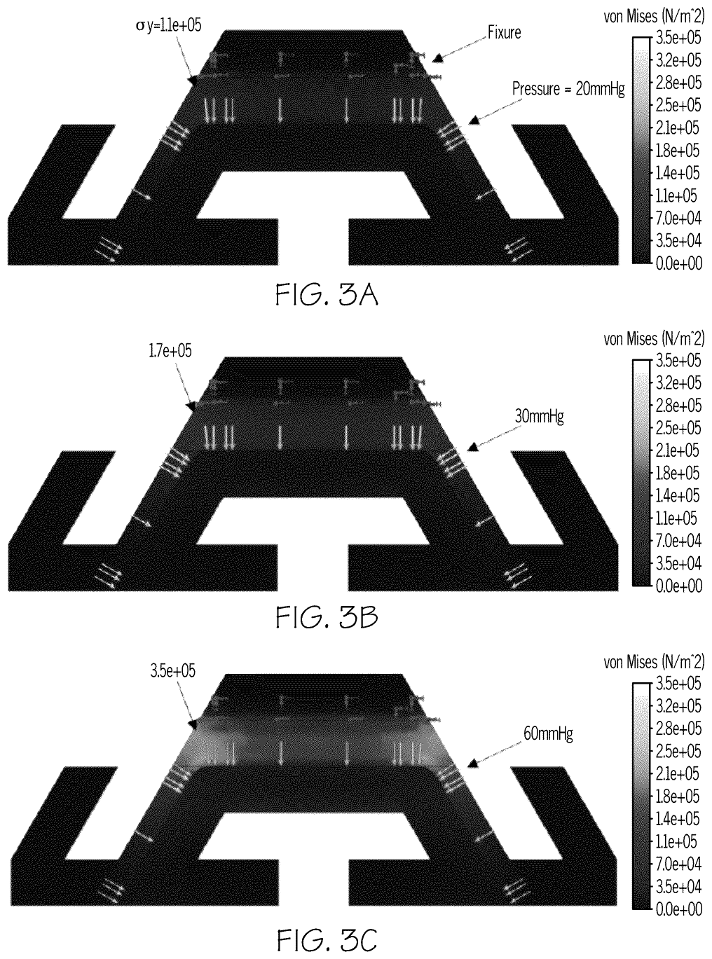

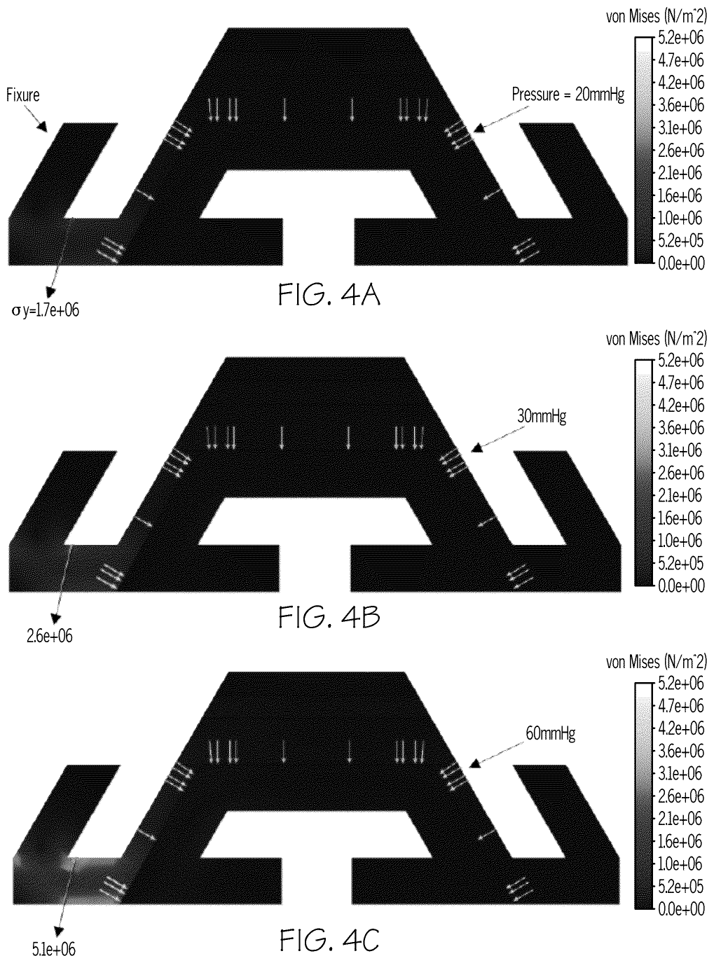

In addition, finite element analysis (FEA) on the LIFT 3D model used in in vivo rodent studies with BioMed White Resin material was performed in Solid Works (Dassault Systèmes SolidWorks Corporation, MA, USA) to understand the effect of stresses and strains induced on the LIFT during the implantation process. The von Mises stress profiles shown in FIGS. 3A-3C and 4A-4C imply that LIFT can withstand the forces applied during the implantation process.

The cured and post-processed LIFT units were assumed to be linear elastic isotropic materials, and the geometry was built with a solid mesh of tetrahedral 3D elements. The simulation study was set up such that the surgeon holds the LIFT at one of the fixture points while the external pressures acting on the LIFT were generated by the nerve placed on the platform and the channels. The external pressures are indicated with red arrows on the LIFT (FIGS. 3A-3C and 4A-4C).

Two fixture points include the fastener receptacles on the ledge (FIGS. 3A-3C) and one of the arms of the reshaping fastener receptacles (FIGS. 4A-4C). Three levels of pressures, low (20 mmHg), optimal (30 mmHg), and extreme pressures (60 mmHg), were modeled. Low pressure can be applied on the nerve chronically without affecting intraneural blood flow, and at extreme pressure, total block of intraneural blood flow.

The von Mises stress profiles show that the maximum stresses are concentrated at the edge of the fastener receptacles (FIGS. 3A-3C) and the intersection point on the arms of the reshaping fastener receptacles (FIGS. 4A-4C). The stresses are higher when the LIFT is held on the reshaping fastener receptacle's arm. However, the maximum stresses estimated on the LIFT during implantation are at least ten times less than the yield stress. Hence, the LIFT fabricated with BioMed White Resin would not fail due to the forces applied during implantation.

Due to limited surgical space and small nerve diameter, the small form factor was necessary for in vivo rodent studies. Hence, the wall thickness of the LIFT was 1 mm, which is what was used for the simulation study. To translate the LIFT design from rat to large animal model and human cadaver studies, the prototypes were fabricated with a 2 mm wall thickness. This change can further increase the stiffness of LIFT.

The design controls were employed to minimize unintentional risk to the nerve during the electrode implantation process. In the nerve supporter feature, the areas where the nerve is placed were smoothed using the “fillet” feature in the CAD design software. The areas where the surgeon grabs to hold the LIFT with a surgical instrument during the implantation were placed sufficiently far from the nerve to avoid inadvertent trauma. In addition, the ledge with fastener receptacles was designed to provide a non-slip grip for microneedle holders or fine tweezers to further avoid any inadvertent nerve trauma due to slippage of the surgical instrument.

The channel width and height design features of the Nerve Shaper are pre-defined for a given nerve diameter to prevent potential nerve trauma. The pre-defined channel's width and height ensure that the cross-sectional area of the channel is greater than the cross-sectional area of the nerve to maintain intraneural pressure and blood flow. Additionally, the minimum height of the channel is greater than the largest fascicle diameter in the nerve to prevent compression injury.

### Example 1.7

### Effectiveness of LIFT

The effectiveness of the LIFT system in its preferred function, to facilitate the implantation process of LIFEs, is demonstrated through 1) in-vivo rodent studies, 2) an in-vivo rabbit study, and 3) translational steps with usability studies.

### Example 1.7.1

### In-vivo Rodent Studies

LIFEs were successfully implanted in the sciatic nerve and elicited nerve responses to the electrical stimulation, proving that LIFT is effective in its intended primary function. In addition, using LIFT during the implantation process significantly reduced the time for implantation. Furthermore, LIFEs implanted with LIFT were stable and functional.

### Example 1.7.2

### In-vivo Rabbit Studies

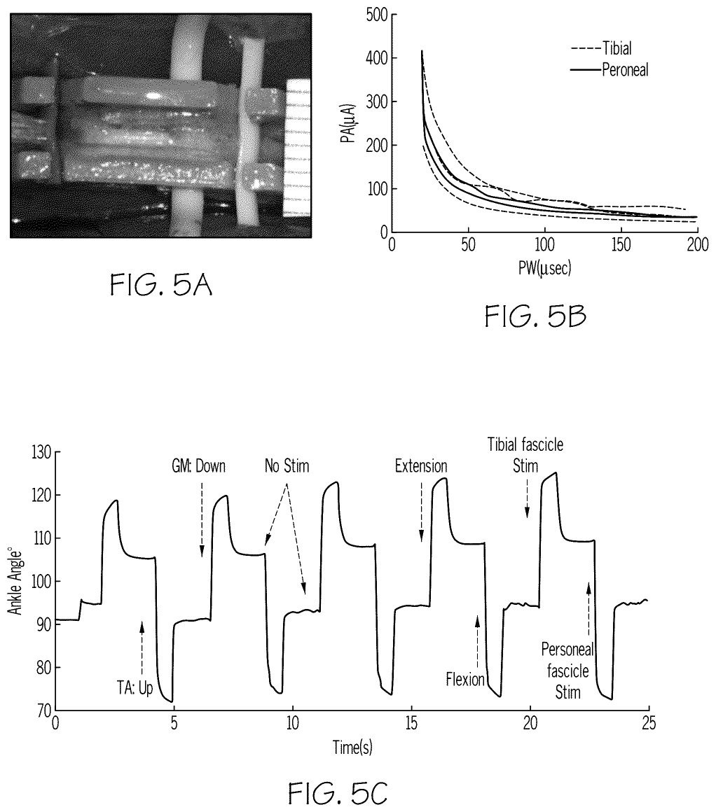

Using a scaled version of LIFT (FIG. 5A), an in-vivo rabbit study was conducted to show the feasibility of implanting LIFEs in the sciatic nerve (larger diameter than rat's sciatic nerve). Two and three LIFEs were implanted in the peroneal and tibial fascicles. Following implantation, strength-duration curves were obtained to determine initial stimulation parameters (FIG. 5B). One LIFE in the tibial fascicle and one LIFE in the peroneal fascicle were chosen for alternating stimulation in order to induce contraction of flexor and extensor muscles that control ankle joint movement (FIG. 5C). Stimulation with a LIFE in the tibial fascicle activated the gastrocnemius muscle (GM) to extend the ankle, and stimulation with a LIFE in the peroneal fascicle activated the tibialis anterior muscle to flex the ankle. These results show that the scaled LIFT permitted the appropriate placement of the LIFE electrodes in the larger nerve in the rabbit animal model,

### Example 1.7.3

### Translational Steps With Usability Studies

In order to translate the LIFTs to use in human clinical studies, human factors were integrated throughout the LIFT development process. Novice users and trained surgeons were asked to use LIFT to thread needles in rat sciatic nerves simulating the implantation of LIFEs. These observations led to incorporating fastener receptacles into the LIFT's design for stabilizing and manipulating the LIFT angle during implanting LIFEs. The new design reduced the possibility of slippage of the LIFT or the surgical instrument, avoiding inadvertent trauma to the nerve.

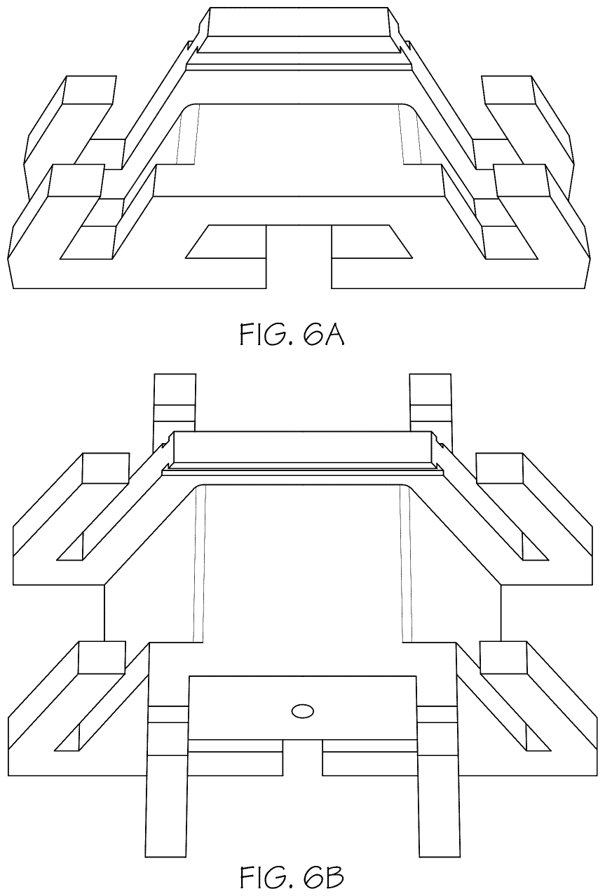

In particular, prior systems for electrode implantation (e.g., the LIFT system shown in FIG. 6A) assisted surgeons in implanting multiple LIFEs effectively and efficiently. However, the intended purpose of the prior tools were to assist surgeons in the implantation process of intraneural electrodes in one or more targeted peripheral nerves in human participants. To translate the use of the LIFT tool from pre-clinical studies to human clinical research studies, LIFT designs were scaled, fabricated, and tested in animal models (rabbits and pigs) with larger-diameter nerves compared to the rodent model. The designs were functional when implemented in rabbits. However, in pig cadaver studies, the designs were not functional because of the loss of tension in the vessel loops that were placed in reshaping fastener receptacles (FIG. 6A).

To address the aforementioned challenges, the LIFT design was modified to include reinforcing fastener receptacles to ensure sustained reshaping of the nerve in the channel. This reinforcing LIFT (ReLIFT) design, as shown in FIG. 6B, enables the implantation of LIFEs in human nerves.

Large-diameter nerves in pig models and humans are naturally oval and consist of multiple fascicles. Hence accessing fascicles that are in the middle of the nerve is challenging. Furthermore, extensive epineural dissection is required to access these fascicles. Reshaping the nerve from an oval to an elongated ellipse distributes the fascicles across the major axis of the elongated ellipse and brings the middle fascicles to the circumference of the ellipse. The reshaping process increases fascicular access for implanting the LIFEs.

It is preferable for a peripheral nerve to remain reshaped during an implantation procedure. To reshape a peripheral nerve, the vessel loops placed in reshaping fastener receptacles must retain tension throughout the procedure. The tension in the vessel loops applies small forces on the nerve nudging inner fascicles to the surface. The tension of the vessel loops held well for the smaller-diameter nerve in rodent studies but did not work for large-diameter nerves in pig cadaver studies. Hence, in the re-designed and modified ReLIFT, reinforcement fastener receptacles were added to sustain the tension in the vessel loops, ensuring nerve reshaping for the larger-diameter nerves in the channel. In addition, the reinforcement fastener receptacles help case implementation by allowing the preloading of the vessel loops in reinforcement fastener receptacles on one side.

The ReLIFT tool is small compared to other surgical instruments used during the implantation surgery. Hence, there is a possibility that the ReLIFT tool may be inadvertently left behind in the body after the implantation process is complete. For example, if the nerve of the target for implantation is in or near the abdominal cavity, then the ReLIFT tool may fall into the abdominal cavity. For these reasons, an aperture (FIG. 6B) was also incorporated in the design to ensure retrieval of the tool using a suture line. This feature introduces safety in device use during the surgical procedure.

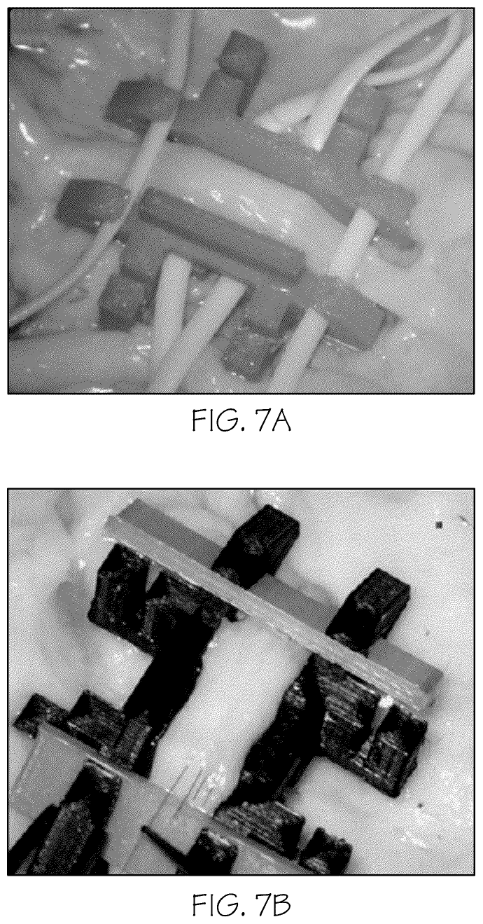

ReLIFT was successfully tested in pig cadaver nerve tissue (FIG. 7A). Further, a vessel loop alternative (FIG. 7B) designed to replace the vessel loop was fabricated and tested in pig cadaver nerve tissue.

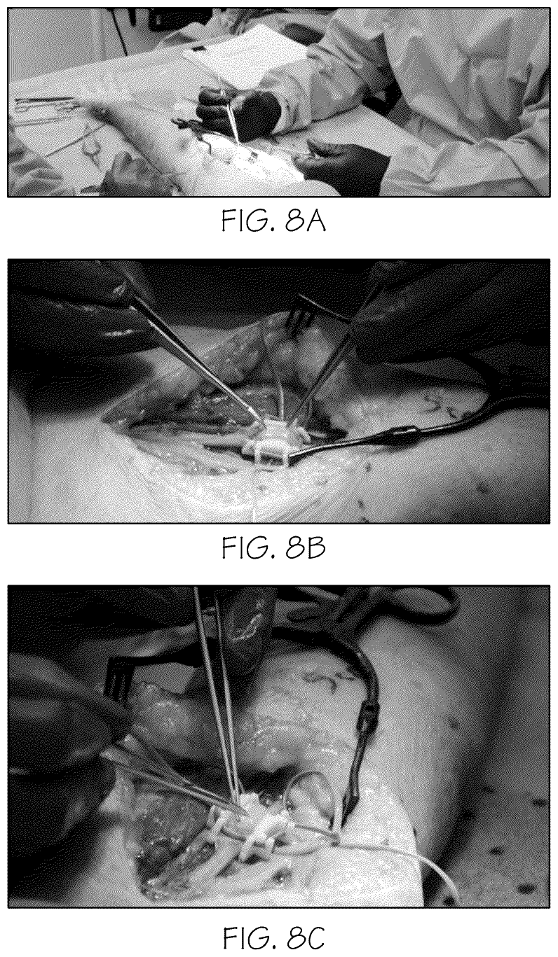

In one human cadaver study, two orthopedic surgeons used ReLIFT to thread needles into the median nerve, as shown in FIGS. 8A-8C. The results from the cadaver study show that ReLIFT could assist surgeons in implanting LIFEs in human nerves.

### Example 1.8

### Discussion

In sum, Applicant has developed a system and method for implanting target-specific multiple intrafascicular electrodes in peripheral nerves to facilitate an implantation process that aims to reduce the difficulty of the implanting procedure and the time taken to implement the procedure. In particular, this Example describes ReLIFT, and a method for utilization of ReLIFT for intrafascicular electrode implantation.

ReLIFT flattens a peripheral nerve to increase access to the number of fascicles available for implantation and supports the nerve during electrode implantation. The intrafascicular electrode implantation method streamlines the implantation procedure.

The ReLIFT system and method were invented to improve the efficiency of delivery and effectiveness of the use of existing intrafascicular electrodes by enhancing the implantation procedure. The ReLIFT system and method are efficient in reducing the complexity of the intrafascicular electrode implantation procedure and in reducing the time taken to implant multiple intrafascicular electrodes.

Additionally, the ReLIFT system and method effectively increase the intrafascicular electrode's inherent selectivity properties. The selectivity is enhanced by providing a way to increase access to the number of fascicles in the nerve during intrafascicular electrode implantation. By using multiple intrafascicular electrodes within a fascicle, further selectivity can be achieved by increasing the set of nerve fibers that are accessible within a fascicle by each intrafascicular electrode.

In sum, the advantages of the ReLIFT system include, without limitation: (1) an increase in implantation selectivity by flattening the peripheral nerve to increase access to the number of fascicles available for implanting by distributing the fascicles along the axis of flattening; (2) a reduction in the invasiveness of the implantation procedure by avoiding extensive epineurial dissection to identify the fascicle; (3) a reduction in the complexity of the implantation process by stabilizing the nerve; (4) a reduction in the time to implant multiple electrodes; and (5) a reduction in the possibility of trauma to the nerve during the electrode implantation process. The advantages of electrode implantation methods that utilize the ReLIFT system include, without limitation, (1) streamlining of the intrafascicular electrode implantation process; and (2) requiring less training in the intrafascicular electrode implantation process.

Without further elaboration, it is believed that one skilled in the art can, using the description herein, utilize the present disclosure to its fullest extent. The embodiments described herein are to be construed as illustrative and not as constraining the remainder of the disclosure in any way whatsoever. While the embodiments have been shown and described, many variations and modifications thereof can be made by one skilled in the art without departing from the spirit and teachings of the invention. Accordingly, the scope of protection is not limited by the description set out above, but is only limited by the claims, including all equivalents of the subject matter of the claims. The disclosures of all patents, patent applications and publications cited herein are hereby incorporated herein by reference, to the extent that they provide procedural or other details consistent with and supplementary to those set forth herein

1. A system for holding at least one peripheral nerve, said system comprising:
a support surface,
wherein the support surface is operational for holding the peripheral nerve;
a first fastener receptacle positioned on the proximal end of the support surface;
a second fastener receptacle positioned on the distal end of the support surface;
a third fastener receptacle positioned on a first side of the support surface; and
a fourth fastener receptacle positioned on a second side of the support surface,
wherein the first and the second side are on opposite sides of one another, and
wherein each fastener receptacle is operational to receive at least one fastener.

2. The system of claim 1, wherein the support surface comprises a flat surface.

3. The system of claim 1, wherein the system further comprises:
a first angled surface positioned on the proximal end of the support surface and behind the first fastener receptacle; and
a second angled surface positioned on the distal end of the support surface and behind the second fastener receptacle.

4. The system of claim 1, wherein each fastener receptacle comprises one or more slots operational to receive a fastener.

5. The system of claim 1, further comprising an aperture, wherein the aperture is positioned below the support surface, wherein the aperture spans the width of the support surface, and wherein the aperture is operational to receive a cord for lifting the system.

6. The system of claim 1, further comprising a base area positioned below the support surface, wherein the base area is operational to stabilize the system on a surface.

7. A method of holding at least one peripheral nerve, said method comprising:
placing a system at or near the peripheral nerve, wherein the system comprises:
a support surface,
a first fastener receptacle positioned on the proximal end of the support surface,
a second fastener receptacle positioned on the distal end of the support surface,
a third fastener receptacle positioned on a first side of the support surface, and
a fourth fastener receptacle positioned on a second side of the support surface, wherein the first and the second side are on opposite sides of one another;
positioning the peripheral nerve on the support surface; and
placing at least one fastener on the first fastener receptacle, the second fastener receptacle, the third fastener receptacle, and the fourth fastener receptacle.

8. The method of claim 7, wherein the positioning of the peripheral occurs prior to placing the at least one fastener on the first fastener receptacle, the second fastener receptacle, the third fastener receptacle, and the fourth fastener receptacle.

9. The method of claim 7, wherein the placing of at least one fastener on the first fastener receptacle, the second fastener receptacle, the third fastener receptacle, and the fourth fastener receptacle secures the peripheral nerve on the system.

10. The method of claim 7, wherein the system further comprises:
a first angled surface positioned on the proximal end of the support surface and behind the first fastener receptacle; and
a second angled surface positioned on the distal end of the support surface and behind the second fastener receptacle.

11. The method of claim 7, wherein the system further comprises an aperture positioned below the support surface, wherein the aperture spans the width of the support surface, and wherein the method further comprises a step of applying a cord through the aperture and pulling the cord to lift the system.

12. The method of claim 7, wherein the peripheral nerve is selected from the group consisting of a brachial plexus, a peroneal nerve, a femoral nerve, a lateral femoral cutaneous nerve, a sciatic nerve, a spinal accessory nerve, a tibial nerve, an autonomic nerve, branches thereof, or combinations thereof.

13. The method of claim 7, wherein the peripheral nerve comprises an autonomic nerve.

14. The method of claim 7, wherein the method facilitates access to one or more fascicles of the peripheral nerve.

15. The method of claim 7, further comprising a step of implanting one or more electrodes into or in between one or more fascicles of the peripheral nerve, wherein the implanting occurs after positioning the peripheral nerve on the support surface.

16. The method of claim 7, wherein the method occurs in vitro.

17. The method of claim 7, wherein the method occurs in vivo in a subject.

18. The method of claim 17, wherein the subject is a human being.

19. The method of claim 7, further comprising a step of repairing the peripheral nerve.
